(19) United States
(12) Patent Application Publication
(10) Pub. No.: US 2016/0244808 A1
(43) Pub. Date: Aug. 25, 2016

(54) CONTAINER, LIQUID STORING MEMBER, CARTRIDGE SET, AND METHOD OF MANUFACTURING LIQUID STORING MEMBER

(71) Applicant: Seiko Epson Corporation, Tokyo (JP)

(72) Inventors: Toshiro MURAYAMA, Fujimi (JP); Kazuhisa MIYASAKA, Suwa (JP)

(21) Appl. No.: 15/048,081

(22) Filed: Feb. 19, 2016

(30) Foreign Application Priority Data
Feb. 20, 2015 (JP) 2015-031625

(51) Int. Cl. C12Q 1/68; B01L 3/00; B65B 7/16

(52) U.S. Cl. CPC C12Q 1/686; B65B 7/161; B01L 3/527; B01L 3/502; C12Q 1/6848; B01L 2300/0832; B01L 2300/044; B01L 2200/16; B01L 2300/0848; B01L 2200/026; B01L 2200/12

(57) ABSTRACT

An elution container is a container that stores a liquid by sealing a first opening. The elution container includes: a first annular wall section having an annular wall surface formed around the first opening; and a first annular attachment surface which is formed on the inner side of the first annular wall section and to which a first film sealing the first opening is attached. The first annular wall section has a height higher than the first attachment surface. In addition, the elution container includes: a second annular wall section having an annular wall surface on a second end portion of the elution container; and a second annular attachment surface which is formed on the inner side of the second annular wall section and to which a second film sealing a second opening is attached. The second annular wall section has a height higher than the second attachment surface.

### BACKGROUND

1. Technical Field

The present invention relates to a container which stores a liquid inside thereof, a liquid storing member in which a liquid is sealed and stored, a cartridge set which enables plural liquid storing members to be bound, and a method of manufacturing the liquid storing member.

2. Related Art

In the field of biochemistry, a technology of a polymerase chain reaction (PCR) has been established. Recently, accuracy in amplification or detection sensitivity in a PCR method has been improved such that it is possible to amplify and detect/analyze an infinitesimal trace of a specimen (DNA or the like). The PCR is a technology in which a thermal cycle is performed on nucleic acids (target nucleic acids) as an amplification target and a solution (reaction solution) including a reagent, and thereby the target nucleic acids are amplified. In general, as the thermal cycle of PCR, a technology, in which the thermal cycle is performed at two-level or three-level temperatures, is employed.

Meanwhile, diagnosis of an infection such as influenza in a field of medical care is mainly performed by using a simple test kit such as an immunochromatography kit in the present circumstances. However, in such a simple test, the test may be performed with insufficient accuracy and it is desirable that the PCR, which can be expected to perform the test with higher accuracy, is applied to diagnosis of an infection.

In recent years, as a device used in the PCR method or the like, a device, in which a water-based liquid layer and a water-insoluble gel layer are alternately stacked in a capillary (in a cartridge), magnetic particles, to which nucleic acids are attached, pass through the layers, and thereby purification of the nucleic acid is performed (see International Publication No. 2012/086243). Also, International Publication No. 2012/086243 discloses that a nucleic acid amplification reaction solution is accommodated in the lowermost layer of the cartridge and amplification of a target nucleic acid in the nucleic acid amplification reaction solution is performed.

However, the device described above is configured to include a container which is integrally formed from a reagent supply section to the nucleic acid amplification reaction solution collecting section. For example, in a case where such a device is kept for a long period of time, a component contained in a cleaning liquid, an eluate, or the like, may be dispersed through oil, which results in contamination, and the PCR may be inhibited. In addition, when outside air infiltrates into a liquid in the device and bubbles are formed, a purification process of the nucleic acids may be inhibited.

### SUMMARY

An advantage of some aspects of the invention is to provide a container in which it is possible to prevent bubbles from being mixed to a liquid when the liquid is stored in the container. Another advantage of some aspects of the invention is to provide a liquid storing member and a method of manufacturing the liquid storing member in which the liquid is sealed and stored while preventing bubbles from being mixed in the liquid. Still another advantage of some aspects of the invention is to provide a cartridge set which enables plural liquid storing members to be bound.

### Application Example 1

A container according to this application example of the invention has an opening and a liquid is sealed and stored therein by sealing the opening, and the container includes: an annular wall section having an annular wall surface formed around the opening; and an attachment surface which is formed on the inner side of the annular wall section and to which a film sealing the opening is attached. The annular wall section has a height higher than the attachment surface.

According to the container related to this application example, since it is possible to attach the film to the attachment surface in a liquid, it is possible to prevent bubbles from being mixed into the liquid.

### Application Example 2

In the container according to the application example of the invention, the attachment surface may be an annular step section formed on a wall surface on the inner side of the annular wall section.

According to the container related to this application example, it is possible to position the film on the wall surface on the inner side and to attach the film to the attachment surface.

### Application Example 3

In the container according to the application example of the invention, the opening, the annular wall section, the film, and the attachment surface are a first opening, a first annular wall section, a first film, and a first attachment surface, respectively, the container may further include: a second opening different from the first opening; a second annular wall section having an annular wall surface formed around the second opening; and a second annular attachment surface which is formed on the inner side of the second annular wall section and to which a second film sealing the second opening is attached, and the second annular wall section may have a height higher than the second attachment surface.

According to the container related to this application example, since it is possible to attach the film to the attachment surface in a liquid on both two openings, it is possible to prevent bubbles from being mixed into the liquid.

### Application Example 4

In the container according to the application example of the invention, the container may have a longitudinal direction, the first opening may be formed in one end portion of the container, and the second opening may be formed in the other end portion of the container.

According to the container related to this application example, this can be applicable to both end openings of the container in the longitudinal direction.

### Application Example 5

A liquid storing member according to this application example of the invention includes the container to which the film is attached to the attachment surface thereof and in which a liquid is sealed and stored.

According to the liquid storing member related to this application example, it is possible to seal and store the liquid in a state in which bubbles are less likely to be mixed.

### Application Example 6

A liquid storing member according to this application example of the invention includes the container to which the first film is attached on the first attachment surface thereof and the second film is attached on the second attachment surface thereof and in which a liquid is sealed and stored between the first film and the second film.

According to the liquid storing member related to this application example, it is possible to seal and store the liquid in a state in which bubbles are less likely to be mixed.

### Application Example 7

A cartridge set according to this application example of the invention includes: the liquid storing member; and another liquid storing member which is bound to the liquid storing member. A liquid is sealed and stored in a first flow path of the liquid storing member and the liquid storing member further includes an insertion section which is inserted into the another liquid storing member. An inside surface of the insertion section forms a part of the first flow path. An opening end of the insertion section is formed at a position lower than the attachment surface. The another liquid storing member has a second flow path that stores another liquid inside, a third annular wall section having an annular wall surface on one end portion side, a third annular attachment surface that is formed on the inner side of the third annular wall section and to which a third film is attached. The third annular wall section has a height higher than the third attachment surface. The third film is attached to the third attachment surface and the another liquid is sealed and stored in the second flow path.

According to the cartridge set related to the application example, it is possible to bind two liquid storing members in which the liquids are sealed and stored in a state in which bubbles are less likely to be mixed. In addition, in this case, it is possible to assemble the cartridge that stores the liquid in a state in which bubbles are less likely to be mixed in a flow path which is formed to communicate with two flow paths.

### Application Example 8

A method of manufacturing a liquid storing member according to this application example of the invention includes: injecting a liquid into the container; filling the container with liquid at a level higher than the attachment surface; and attaching the film to the attachment surface in the liquid to seal and store the liquid.

According to the method of manufacturing a liquid storing member related to this application example, the film is attached in the liquid, and thereby it is possible to prevent bubbles from being mixed into the liquid sealed and stored.

### BRIEF DESCRIPTION OF THE DRAWINGS

The invention will be described with reference to the accompanying drawings, wherein like numbers reference like elements.

FIG. 1 is a front view of a container assembly according to an embodiment.

FIG. 2 is a side view of the container assembly according to the embodiment.

FIG. 3 is a plan view of the container assembly according to the embodiment.

FIG. 4 is a perspective view of the container assembly according to the embodiment.

FIG. 5 is a sectional view of the container assembly according to the embodiment, which is taken along line A-A in FIG. 3.

FIG. 6 is a sectional view of the container assembly according to the embodiment, which is taken along line C-C in FIG. 3.

FIGS. 7A and 7B are views schematically illustrating an operation of the container assembly according to the embodiment.

FIGS. 8A and 8B are views schematically illustrating an operation of the container assembly according to the embodiment.

FIG. 9 is a diagram of a schematic configuration of a PCR device.

FIG. 10 is a block diagram of the PCR device.

FIG. 11 is a perspective view of an elution container according to an embodiment.

FIG. 12 is a sectional view of a part of the elution container on a first end portion, which is taken along line B-B.

FIG. 13 is a sectional view of a part of the elution container on a second end portion, which is taken along line B-B.

FIG. 14 is a perspective view of a reaction container according to an embodiment.

FIG. 15 is a sectional view of apart of the reaction container on a third end portion side, which is taken along line B-B.

FIG. 16 is a sectional view of a cartridge set according to an embodiment, which is taken along line B-B.

FIG. 17 is a sectional view of a part of a nuclei acid amplifying reaction cartridge, which is taken along line B-B.

### DESCRIPTION OF EXEMPLARY EMBODIMENTS

Hereinafter, preferred embodiments of the invention will be described in detail with reference to the drawings. Further, the embodiments to be described below do not inappropriately limit the content of the invention described in the appended claims. In addition, every configuration to be described below is not essential requirements of the invention.

A container according to the present embodiment has an opening and a liquid is sealed and stored therein by sealing the opening. The container includes: an annular wall section having an annular wall surface formed around the opening; and an annular attachment surface which is formed on the inner side of the annular wall section and to which a film sealing the opening is attached. The annular wall section has a height higher than the attachment surface.

In a liquid storing member according to the present embodiment, a liquid is sealed and stored with the film attached to the attachment surface of the container.

A cartridge set according to the present embodiment includes: the liquid storing member; and another liquid storing member which is bound to the liquid storing member. A liquid is sealed and stored in a first flow path of the liquid storing member and the liquid storing member further includes an insertion section which is inserted into the another liquid storing member. An inside surface of the insertion section forms a part of the first flow path. An opening end of the insertion section is formed at a position lower than the attachment surface. The another liquid storing member has a second flow path that stores another liquid inside, a third annular wall section having an annular wall surface on one end portion side, a third annular attachment surface that is formed on the inner side of the third annular wall section and to which a third film is attached. The third annular wall section has a height higher than the third attachment surface. The third film is attached to the third attachment surface and the another liquid is sealed and stored in the second flow path.

A method of manufacturing a liquid storing member according to the present embodiment includes: injecting a liquid into the container; filling the container with the liquid at a level higher than the attachment surface; and attaching the film to the attachment surface in the liquid to seal and store the liquid.

For the cartridge set according to the invention, a set for assembling cartridges, in which a nucleic acid amplification reaction is performed, is described. In other words, when a nucleic acid amplification reaction cartridge set according to the invention is assembled, it is possible to obtain a cartridge for performing a nucleic acid amplification reaction. Hereinafter, the cartridge (container assembly) is, first, described, and then the container, the liquid storing member, a method of manufacturing the liquid storing member, and the nucleic acid amplification reaction cartridge set will be described.

### 1. Outline of Container Assembly

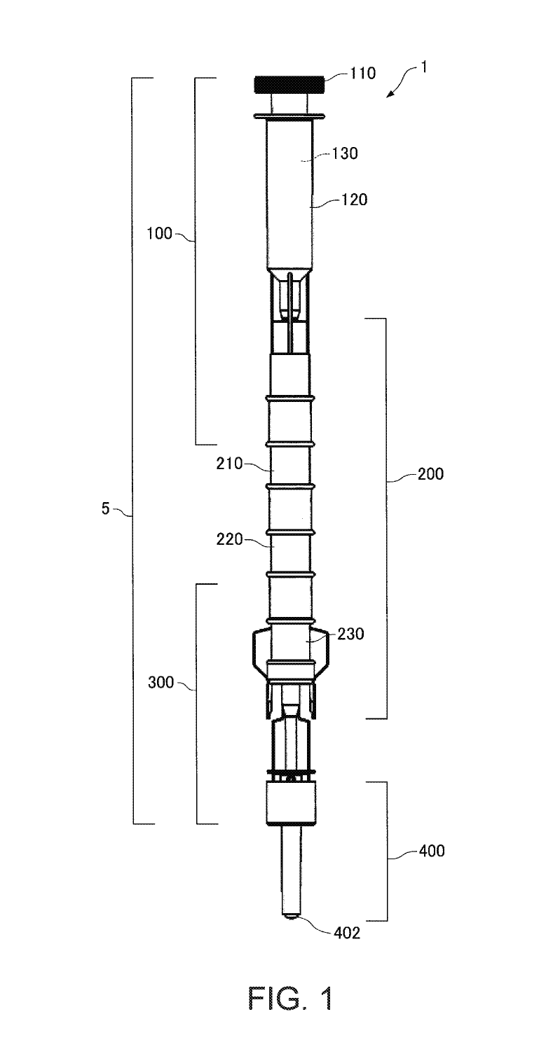

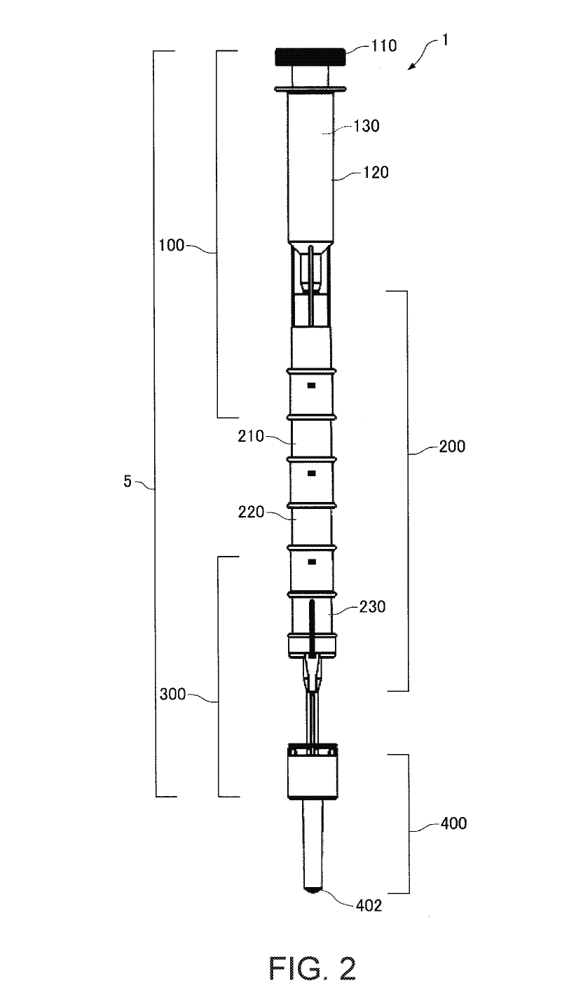

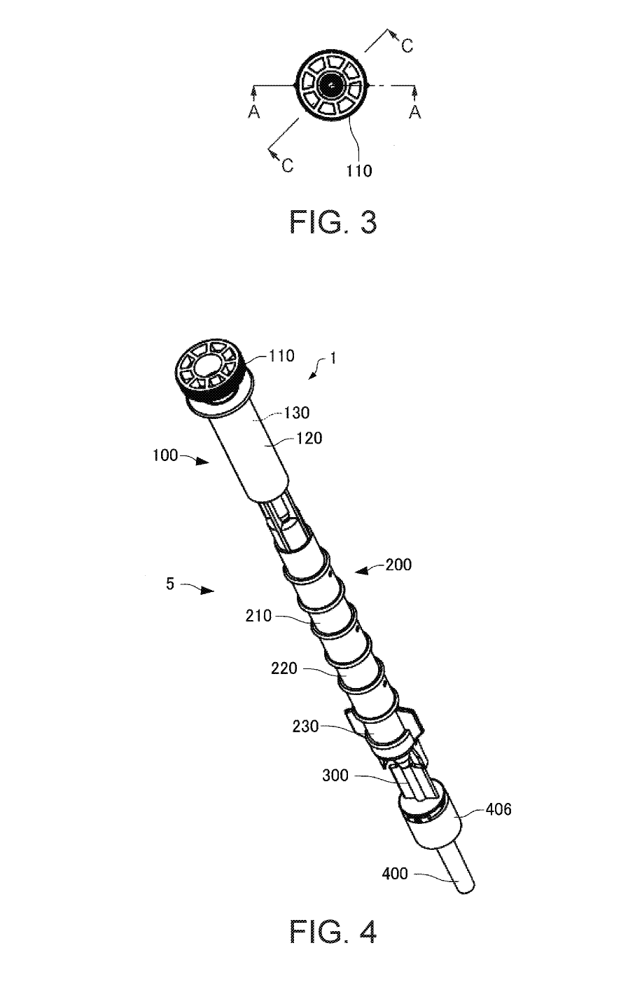

First, an outline of a container assembly 1 according to the present embodiment is described with respect to FIG. 1 to FIG. 4. FIG. 1 is a front view of the container assembly 1 according to the embodiment (hereinafter may be referred to as a cartridge). FIG. 2 is a side view of the container assembly 1 according to the embodiment. FIG. 3 is a plan view of the container assembly 1 according to the embodiment. FIG. 4 is a perspective view of the container assembly 1 according to the embodiment. Further, a state of the container assembly 1 in FIG. 1 to FIG. 3 is described as an upright state.

The container assembly 1 includes an adsorption container 100, a cleaning container 200, an elution container 300, and a reaction container 400. The container assembly 1 is a container forming a flow path (not illustrated) through which communication from the adsorption container 100 to reaction container 400 is performed. One end of the flow path of the container assembly 1 is closed by a cap 110 and the other end thereof is closed by a bottom 402.

In the container assembly 1, preprocessing of combining nucleic acids with a magnetic bead (not illustrated) in the adsorption container 100, purifying the nucleic acids with the magnetic bead moving in the cleaning container 200, and eluting the nucleic acids in an eluate droplet (not illustrated) in the elution container 300, and thermal cycle processing of a polymerase reaction to the eluate droplet containing the nucleic acids in the reaction container 400 are performed.

A material for the container assembly 1 is not particularly limited; however, it is possible to use, for example, glass, a polymer, metal, or the like. It is more preferable that a material such as glass or the polymer, which has transparency in the visible light, is selected as the material of the container assembly 1, because it is possible to observe the inside (cavity) of the container assembly 1 from the outside thereof. In addition, it is preferable that a material, which transmits a magnetic force, or a nonmagnetic material is selected as the material of the container assembly 1, because it is easy to pass the magnetic bead (not illustrated) through the container assembly 1 by applying a magnetic force from the outside of the container assembly 1. For the material of the container assembly 1, it is possible to use a polypropylene resin.

The adsorption container 100 includes a cylindrical syringe section 120 which accommodates an adsorption solution (not illustrated) inside, a plunger section 130 which a movable plunger inserted into the inside of the syringe section 120, and a cap 110 fixed to one end portion of the plunger section 130. In the adsorption container 100, the cap 110 moves to the syringe section 120 such that the plunger section 130 slides on the inner surface of the syringe section 120, and it is possible to extrude the adsorption solution (not illustrated), which is accommodated in the syringe section 120, to the cleaning container 200. Further, the adsorption solution will be described below.

The cleaning container 200 is obtained by binding and assembling first to third cleaning containers 210, 220 and 230. The first to third cleaning containers 210, 220 and 230 have one or more cleaning solution layers partitioned by an oil layer (not illustrated) inside. Also the first to third cleaning containers 210, 220 and 230 are bound, and thereby the cleaning container 200 has a plurality of cleaning solution layers partitioned by a plurality of oil layers (not illustrated) inside. In the cleaning container 200 of the present embodiment, an example, in which three cleaning containers of the first to third cleaning containers 210, 220 and 230 are used, is described; but the number of cleaning containers is not limited thereto, but the number of cleaning containers is appropriately increased or decreased. The cleaning solution will be described below.

The elution container 300 is bound to the third cleaning container 230 of the cleaning container 200 and the eluate is accommodated inside in a state of maintaining a plug shape. Here, the “plug” means a liquid in a case where a specific liquid occupies a zone in the flow path. More specifically, the plug of the specific liquid indicates that only the specific liquid substantially occupies the inside to have a column shape and represents a state in which a certain space inside the flow path is demarcated by the plug. Here, the expression, substantially, indicates that a trace (for example, a thin film shape) of other substances (liquid or the like) may exist around the plug, that is, on the inside wall of the flow path. Further, the eluate will be described below.

A nucleic acid purifying device 5 includes the adsorption container 100, the cleaning container 200, and the elution container 300.

The reaction container 400 is a container which is bound to the elution container 300 and receives a liquid extruded from the elution container 300 and a container that accommodates an eluate droplet containing a specimen during the thermal cycle processing. In addition, the reaction container 400 accommodates a reagent (not illustrated). Further, the reagent will be described below.

### 2. Detailed Structure of Container Assembly

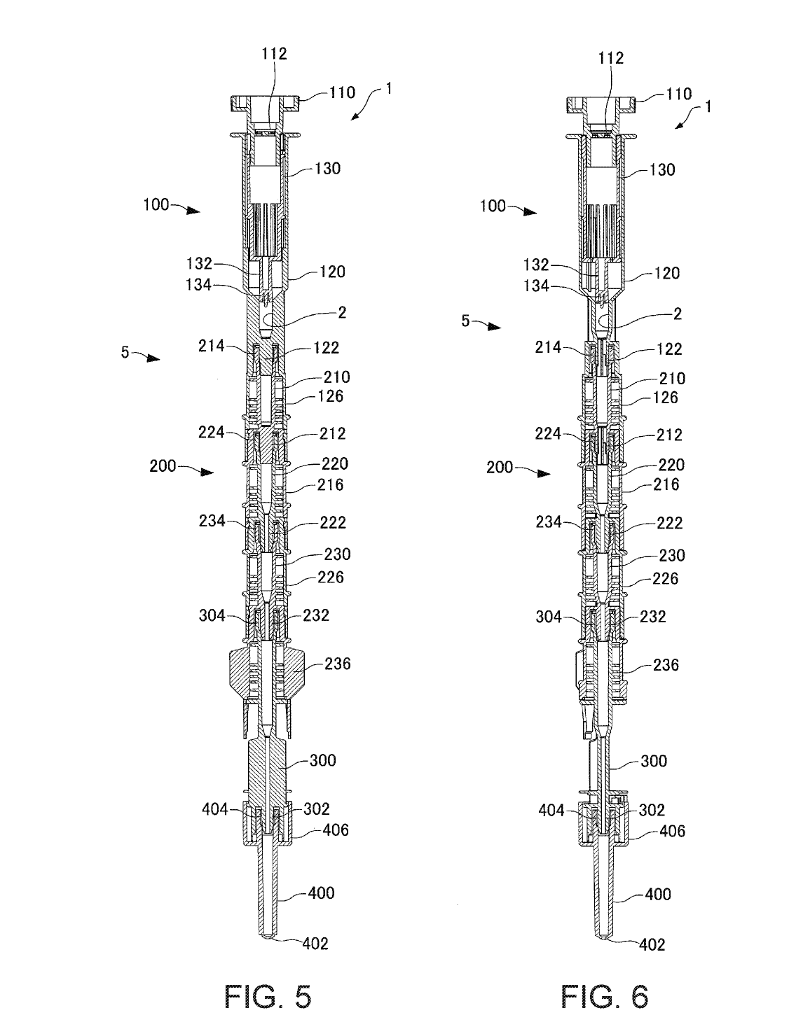

Next, a detailed structure of the container assembly 1 will be described with reference to FIG. 5 and FIG. 6. FIG. 5 is a sectional view of the container assembly 1 according to the embodiment, which is taken along line A-A in FIG. 3. FIG. 6 is a sectional view of the container assembly 1 according to the embodiment, which is taken along line C-C in FIG. 3. Further, actually, the container assembly 1 is assembled in a state of being filled with content such as the cleaning solution; however, in order to describe the structure of the container assembly 1, depiction of the content is omitted in FIG. 5 and FIG. 6.

### 2-1. Adsorption Container

In the adsorption container 100, the plunger section 130 is inserted from one opening end portion of the syringe section 120 and the cap 110 is inserted into an opening end portion of the plunger section 130. The cap 110 has a vent section 112 at the center thereof and it is possible to suppress a change in an internal pressure of the plunger section 130 by the vent section 112 when the plunger section 130 is operated.

The plunger section 130 is a substantially cylindrical plunger which slides on the inner circumferential surface of the syringe section 120 and has the opening end portion into which the cap 110 is inserted, a rod-shaped section 132 which extends from the bottom facing the opening end portion, in the longitudinal direction of the syringe section 120, and a distal end portion 134 which is the distal end of the rod-shaped section 132. The rod-shaped section 132 protrudes from the center of the bottom of the plunger section 130, and a through-hole is formed on the periphery of the rod-shaped section 132 and communicates with the plunger section 130 and the syringe section 120.

The syringe section 120 constitutes a part of a flow path 2 of the container assembly 1 and has a large-diameter section which accommodates the plunger section 130, a small-diameter section smaller in size than the large-diameter section, a diameter-reduction section at which the inner diameter is reduced from the large-diameter section to the small-diameter section, an adsorption inserting section 122 on the distal end of the small-diameter section, and a cylindrical adsorption covering section 126 which covers the periphery of the adsorption inserting section 122. The large-diameter section, the small-diameter section, and the adsorption inserting section 122, as a part of the flow path 2 of the container assembly 1 have substantially a cylindrical shape.

At the time of being provided to an operator, the distal end portion 134 of the plunger section 130 seals the small-diameter section of the syringe section 120, the large-diameter section and the diameter-reduction section are divided from the small-diameter section, and thus two zones are formed.

The adsorption inserting section 122 of the syringe section 120 is inserted and fitted into a first reception section 214 which is one end opening portion of the first cleaning container 210 in the cleaning container 200, and thereby the syringe section 120 and the first cleaning container 210 are bound. The outer circumferential surface of the adsorption inserting section 122 and the inner circumferential surface of the first reception section 214 are brought into close contact with each other and the liquid as the content is prevented from being leaked to the outside.

### 2-2. Cleaning Container

The cleaning container 200 constitutes apart of the flow path 2 of the container assembly 1 and is an assembly of the first to third cleaning containers 210, 220 and 230. Since the first to third cleaning containers 210, 220 and 230 have the same fundamental structure, the structure of the first cleaning container 210 is described and description of the second and third cleaning containers 220 and 230 is omitted.

The first cleaning container 210 has substantially a cylindrical shape extending in the longitudinal direction of the container assembly 1, and includes a first insertion section 212 formed on one opening end portion, the first reception section 214 formed on the other opening end portion, and a cylindrical first covering section 216 which covers the periphery of the first insertion section 212.

The outer diameter of the first insertion section 212 is substantially equal to the inner diameter of the second reception section 224. In addition, the inner diameter of the first reception section 214 is substantially equal to the outer diameter of the adsorption inserting section 122.

The first insertion section 212 of the first cleaning container 210 is inserted and fitted into the second reception section 224 of the second cleaning container 220, and thereby the outer circumference of the first insertion section 212 and the inner circumference of the second reception section 224 are brought into close contact and sealed with each other, and the first cleaning container 210 and the second cleaning container 220 are bound. Similarly, the first to third cleaning containers 210, 220 and 230 are interconnected and the cleaning container 200 is formed. Here, “sealing” means blocking such that at least a liquid or a gas accommodated in the container or the like does not leak to the outside and may include blocking a liquid or a gas from infiltrating into the inside from the outside.

### 2-3. Elution Container

The elution container 300 has substantially a cylindrical shape extending in the longitudinal direction of the container assembly 1 and constitutes a part of the flow path 2 of the container assembly 1. The elution container 300 has an elution inserting section 302 formed on one opening end portion and an elution receiving section 304 formed on the other opening end portion.

The inner diameter of the elution receiving section 304 is substantially equal to the outer diameter of a third insertion section 232 of the third cleaning container 230. The third insertion section 232 is inserted and fitted into the elution receiving section 304, and thereby the outer circumference of the third insertion section 232 and the inner circumference of the elution receiving section 304 are brought into close contact and sealed with each other, and the third cleaning container 230 and the elution container 300 are bound.

### 2-4. Reaction Container

The reaction container 400 has substantially a cylindrical shape extending in the longitudinal direction of the container assembly 1 and constitutes a part of the flow path 2 of the container assembly 1. The reaction container 400 has a reaction receiving section 404 formed on one opening end portion, a bottom 402 formed on the other closed end portion, and a reservoir section 406 that covers the reaction receiving section 404.

The inner diameter of the reaction receiving section 404 is substantially equal to the outer diameter of the elution inserting section 302 of the elution container 300. The elution inserting section 302 is inserted and fitted into the reaction receiving section 404, and thereby the elution container 300 and the reaction container 400 are bound.

The reservoir section 406 having a predetermined space is provided on the periphery of the reaction receiving section 404. The reservoir section 406 has a volume to receiving the liquid overflowing from the reaction container 400 due to movement of the plunger section 130.

### 3. Content of Container Assembly and Operation of Container Assembly

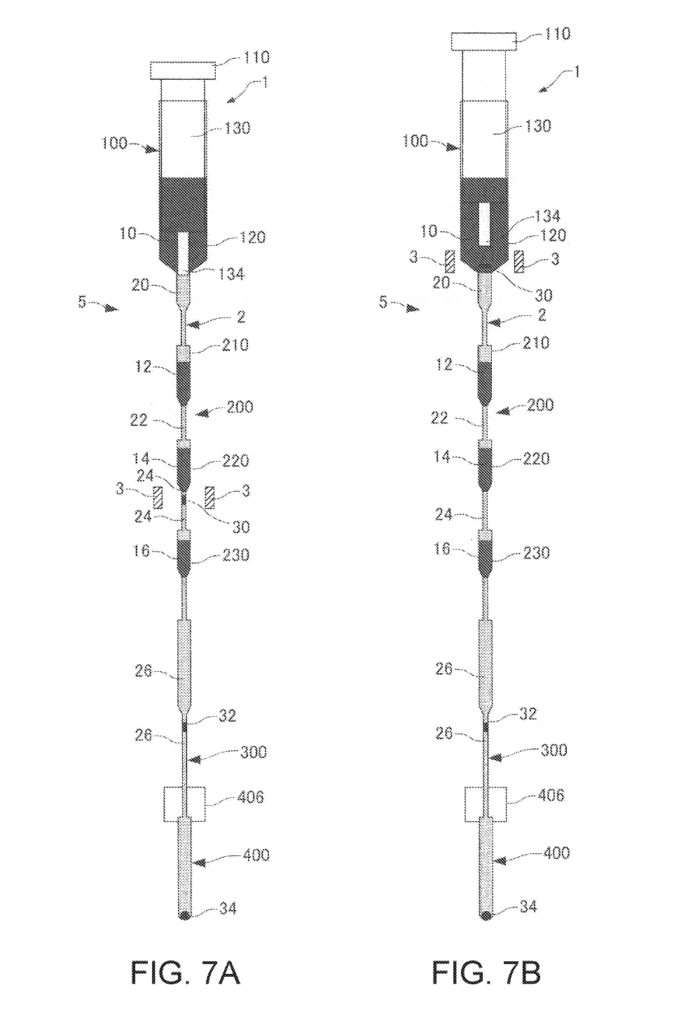

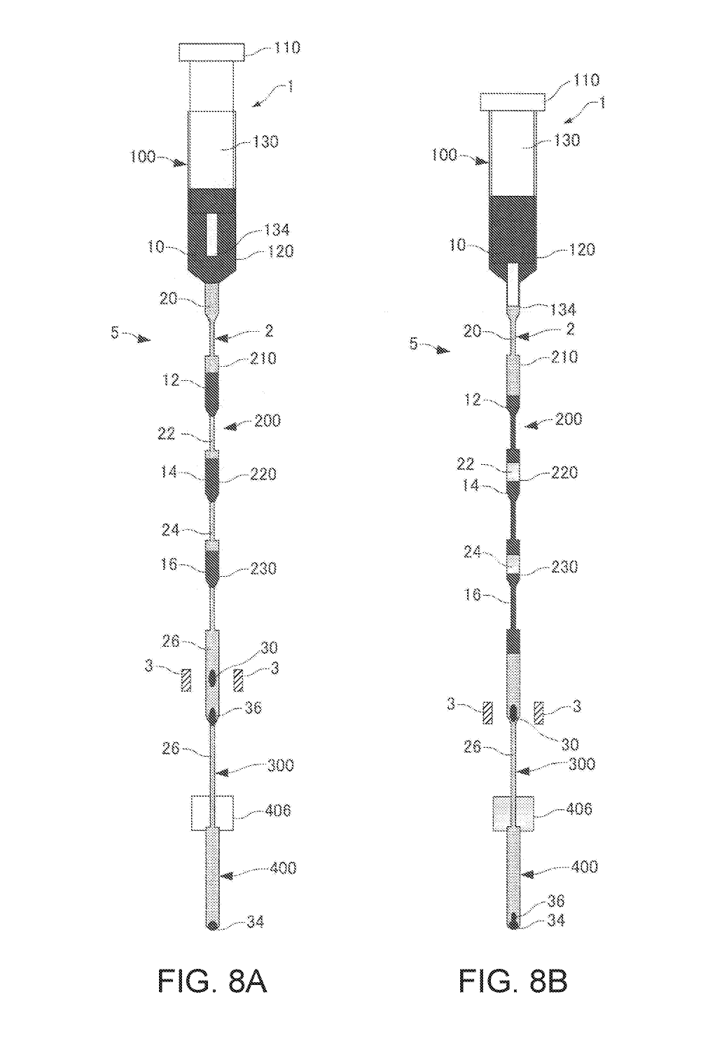

Next, the content in the container assembly 1 will be described with reference to FIG. 7A and an operation of the container assembly 1 will be described with reference to FIG. 7A to FIG. 8B. FIGS. 7A and 7B are views schematically illustrating the operation of the container assembly 1 according to the embodiment. FIGS. 8A and 8B are views schematically illustrating the operation of the container assembly 1 according to the embodiment. Further, in order to depict a state of the content, the respective containers are depicted as the flow path 2 and the external appearance and a binding structure are omitted in FIG. 7A to FIG. 8B.

### 3-1. Content

FIG. 7A illustrates a state of the content in the flow path 2 in the state in FIG. 1. The content in the flow path 2 is, in order from the cap 110 side to the reaction container 400, an adsorption solution 10, a first oil 20, a first cleaning solution 12, a second oil 22, a second cleaning solution 14, a third oil 24, a magnetic bead 30, a third oil 24, a third cleaning solution 16, a fourth oil 26, an eluate 32, a fourth oil 26, and a reagent 34.

In the flow path 2, a portion (thick portion of the flow path 2) having a large cross-sectional area of a plane orthogonal to the longitudinal direction of the container assembly 1 and a portion (slim portion of the flow path 2) having a small cross-sectional area are alternately disposed. Apart or all of the first to fourth oils 20, 22, 24, and 26 and the eluate 32 are accommodated in the thin portions of the flow path 2. The cross-sectional area of the thin portion of the flow path 2 is an area in which, in a case where an interface between liquids (hereinafter, including a fluid) which are adjacent and not mixed to each other is disposed in the thin portion of the flow path 2, the interface can be stably maintained. Accordingly, the liquid disposed in the thin portion of the flow path 2 enables a positional relationship between the liquid and the other liquids disposed on the upper and lower sides of the liquids to be stably maintained. In addition, even in a case where an interface between the liquid disposed in the thin portion of the flow path 2 and the other liquid disposed in the thick portion of the flow path 2 is formed in the thin portion of the flow path 2, and the interface is stirred due to an impact, the flow path is placed in a stationary state and the interface is stably formed at a predetermined portion.

The thin portions of the flow path 2 are formed on the inner sides of the adsorption inserting section 122, the first insertion section 212, the second insertion section 222, the third insertion section 232, and the elution inserting section 302, and extend upward over the elution inserting section 302 in the elution container 300. Further, the liquids accommodated in the thin portions of the flow path 2 are stably maintained even before the container is assembled.

### 3-1-1. Oil

The first to fourth oils 20, 22, 24, and 26 are all formed of oils and exist as the plugs between the liquids before and after the respective oils in the state in FIGS. 7A and 7B. In order for the first to fourth oils 20, 22, 24, and 26 to exist as the plugs, as the liquids adjacent before and after the respective oils, liquids which are phase-separated from each other, that is, liquids which are not mixed, are selected. In addition, the oil constituting the first to fourth oils 20, 22, 24, and 26 may be different types of oils. The oil used for the first to fourth oils can be selected from, for example, silicone-based oil such as dimethyl silicone oil, paraffinic oil, mineral oil, and a compound thereof.

### 3-1-2. Adsorption Solution

The adsorption solution 10 indicates a liquid in which the nucleic acids are adsorbed to the magnetic bead 30, for example, an aqueous solution containing a chaotropic agent. As the adsorption solution 10, 5 M of guanidine thiocyanate, 2% of Triton X-100, or 50 mM of Tris-HCl (pH 7.2) can be used. As long as the adsorption solution 10 contains the chaotropic agent, there is no particular limitation to the adsorption solution; however, the adsorption solution 10 may contain a surfactant in order to break a cell membrane or to denature protein contained in a cell. As long as the surfactant is, in general, used for extracting the nucleic acids from a cell, or the like, there is no particular limitation to the surfactant; however, specifically, examples of the surfactant include a nonionic surfactant like a triton-based surfactant such as Triton-X or a tween-based surfactant such as Tween 20, an anionic surfactant such as N-Lauroylsarcosine sodium (SDS); however, particularly, it is preferable that nonionic surfactant is used in a range of 0.1% to 2%. Further, it is preferable that a reducing agent such as 2-mercaptoethanol or dithiothreitol is contained. A solution may be a buffer solution, and preferably a neutral solution with pH 6 to pH 8. In this respect, specifically, it is preferable that 3 M to 7 M of guanidine salt, 0% to 5% of a nonionic surfactant, 0 mM to 0.2 mM of EDTA, 0 M to 0.2 M of the reducing agent, or the like is contained.

Here, the chaotropic agent generates a chaotropic ion (monovalent anion having a large ion radius) in the aqueous solution and increases water solubility of a hydrophobic molecule. As long as the chaotropic agent contributes to adsorption of the nucleic acids to a solid-phase support, there is no particular limitation to the chaotropic agent. Specifically, examples of the chaotropic agent include guanidinium hydrochloride, sodium iodide, sodium perchlorate, or the like; however, it is preferable that guanidine thiocyanate or guanidinium hydrochloride, which actively denatures the protein, is used. The concentration of the chaotropic agent in a specification varies depending on the respective substances. For example, it is preferable that in a case where guanidine thiocyanate is used, the concentration is in a range of 3 M to 5.5 M and in a case where guanidinium hydrochloride is used, the concentration is equal to or more than 5 M.

The chaotropic agent exists in the aqueous solution, and thereby it is thermodynamically more advantageous that the nucleic acids in the aqueous solution are adsorbed on a solid than exist to be surrounded by water molecules, the nucleic acids are absorbed to the surface of the magnetic bead 30.

### 3-1-3. Cleaning Solution

The first to third cleaning solutions 12, 14, and 16 clean the magnetic bead 30 with which the nucleic acids are combined.

The first cleaning solution 12 is a liquid which is phase-separated from both the first oil 20 and the second oil 22. The first cleaning solution 12 is preferably water or a low salt concentration aqueous solution, and preferably, a buffer solution in the case of the low salt concentration aqueous solution. Salt concentration of the low salt concentration aqueous solution is preferably equal to or lower than 100 mM, more preferably equal to or lower than 50 mM, and most preferably equal to or lower than 10 mM. In addition, the first cleaning solution 12 may contain the surfactant as described above, and there is no particular limitation to pH. In order to use the buffer solution as the first cleaning solution 12, there is no particular limitation to the salt; however, it is preferable that Tris, HEPES, PIPES, phosphoric acid, or the like, is used. Further, it is preferable that the first cleaning solution 12 is contained by an amount with which adsorption of alcohol to a support of the nucleic acids, a reverse transfer reaction, the PCR reaction, or the like is not inhibited. In this case, there is no particular limitation to the concentration of the alcohol.

Further, the chaotropic agent may be contained in the first cleaning solution 12. For example, when the guanidinium hydrochloride is contained in the first cleaning solution 12, it is possible to clean the magnetic bead 30 or the like which maintains or strengthens adsorption of the nucleic acids which are adsorbed to the magnetic bead 30 or the like.

The second cleaning solution 14 is a liquid which is phase-separated from both the second oil 22 and the third oil 24. The second cleaning solution 14 may have a composition which is the same as or different from that of the first cleaning solution 12; however, it is preferable that a solution, which virtually does not contain the chaotropic agent, is used. This is because the chaotropic agent does not infiltrate in the solution adjacent to the cleaning solution. As the second cleaning solution 14, for example, 5 mM of Tris-HCl buffer may be used. As described above, it is preferable that the second cleaning solution 14 contains alcohol.

The third cleaning solution 16 is a liquid which is phase-separated from both the third oil 24 and the fourth oil 26. Basically, the third cleaning solution 16 may have a composition which is the same as or different from that of the second cleaning solution 14; however, the cleaning solution does not contain alcohol. In addition, the third cleaning solution 16 can contain citric acid in order to prevent the alcohol from entering the reaction container 400.

### 3-1-4. Magnetic Beads

The magnetic bead 30 is a bead which adsorbs the nucleic acids and it is preferable to have relatively strong magnetism such that a magnet 3 positioned outside the container assembly 1 causes the magnetic bead to move. The magnetic bead 30 may be, for example, a silica bead or a silica-coated bead. The magnetic bead 30 may be, preferably, the silica-coated bead.

### 3-1-5. Elution Solution

The eluate 32 is a liquid which is phase-separated from the fourth oil 26 and exists as a plug interposed between the fourth oils 26 and 26 in the flow path 2 in the elution container 300. The eluate 32 is a liquid which elutes the nucleic acids adsorbed to the magnetic bead 30, into the eluate 32 from the magnetic bead 30. In addition, the eluate 32 forms a droplet in the fourth oil 26 through heating. As the eluate 32, for example, pure water can be used. Here, the “droplet” means a liquid surrounded by a free surface.

### 3-1-6. Reagent

The reagent 34 contains a component required for reaction. In a case where the reaction in the reaction container 400 is the PCR, it is possible for the reagent 34 to contain at least one of enzymes and a primer (nucleic acid) such as a DNA polymerase for amplifying target nucleic acids (DNA) eluted in a droplet 36 (refer to FIGS. 8A and 8B) of the eluate, and a fluorescent probe that detects an amplified product. Here, all of the primer, the enzyme, and the fluorescent probe are contained. The reagent 34 is incompatible with the fourth oil 26 and is melted, is reacted when the reagent comes into contact with the droplet 36 of the eluate 32 which contains the nucleic acid, and exists in the lowermost region in a gravity direction of the flow path 2 in the reaction container 400 in a solid state. For example, the reagent 34 can use lyophilization (freeze-drying).

### 3-2. Operation of Container Assembly

An example of an operation of the container assembly 1 is described with respect to FIG. 7A to FIG. 8B.

The operation of the container assembly 1 includes (A) a process of assembling the container assembly 1 by binding the adsorption container 100, the cleaning container 200, the elution container 300, and the reaction container 400, (B) a process of guiding a specimen containing the nucleic acids to the adsorption container 100 in which the adsorption solution 10 is accommodated, (C) a process of moving of the magnetic bead 30 from the second cleaning container 220 to the adsorption container 100, (D) a process of oscillating the adsorption container 100 and adsorbing the nucleic acids to the magnetic bead 30, (E) a process of moving of the magnetic bead 30, to which the nucleic acids are adsorbed, to the elution container 300 from the adsorption container 100 through the first oil 20, the first cleaning solution 12, the second oil 22, the second cleaning solution 14, the third oil 24, the third cleaning solution 16, and the fourth oil 26, in this order, (F) a process of eluting the nucleic acids from the magnetic bead 30 into the eluate 32 in the elution container 300, and (G) a process of causing the droplet containing the nucleic acids to come into contact with the reagent 34 in the reaction container 400.

Hereinafter, the respective process will be described in the order.

### (A) Process of Assembling Container Assembly 1

As illustrated in FIG. 7A, in the process of assembly, the adsorption container 100 to the reaction container 400 are bound and the container assembly 1 is assembled such that the flow path 2 which is continuous from the adsorption container 100 to the reaction container 400. Further, in FIG. 7A, the cap 110 is mounted in the adsorption container 100; however, the cap 110 is mounted on the plunger section 130 after (B) process.

More specifically, the elution inserting section 302 of the elution container 300 is inserted into the reaction receiving section 404 of the reaction container 400, the third insertion section 232 of the third cleaning container 230 is inserted into the elution receiving section 304 of the elution container 300, the second insertion section 222 of the second cleaning container 220 is inserted into the third reception section 234 of the third cleaning container 230, the first insertion section 212 of the first cleaning container 210 is inserted into the second reception section 224 of the second cleaning container 220, and the adsorption inserting section 122 of the adsorption container 100 is inserted into the first reception section 214 of the first cleaning container 210.

### (B) Process of Guiding Specimen

The process of guiding is performed by putting a cotton swab, to which, for example, a specimen is attached, in the adsorption solution 10 from the opening in which the cap 110 of the adsorption container 100 is mounted, and immersing the cotton swab in the adsorption solution 10. More specifically, the cotton swab is put in from the opening as one end portion of the plunger section 130 which is in a state of being inserted into the syringe section 120 of the adsorption container 100. Next, the cotton swab is taken out from the adsorption container 100 and the cap 110 is mounted. The state described above is shown in FIG. 7A. In addition, the specimen may be guided to the adsorption container 100 by using a pipette, or the like. In addition, if the specimen is in a paste state or a solid state, for example, the specimen may be attached or may be input to the inner wall of the plunger section 130 to the adsorption container 100 by means of a spoon or tweezers. As illustrated in FIG. 7A, the syringe section 120 and the plunger section 130 are filled with the adsorption solution 10 to an intermediate position; however, a space remains on the opening side on which the cap 110 is mounted.

The nucleic acids as the target are contained in the specimen. Hereinafter, the specimen is simply referred to as the target nucleic acids. For example, the target nucleic acids are DNA or RNA (DNA: deoxyribonucleic acid, and/or RNA: Ribonucleic acid). The target nucleic acids are used as a template of the PCR after the target nucleic acids are extracted from the specimen and are eluted to the eluate 32 to be described. Examples of the specimen include blood, nasal mucus, oral mucosa and other various biological samples.

### (C) Process of Moving of Magnetic Bead

The process of moving of the magnetic bead 30 is performed by causing the magnet 3 to move toward the adsorption container 100 in a state in which a magnetic force of the magnet 3 disposed outside the container is applied to the magnetic bead 30 which exists to have the plug shape by being interposed between the third oils 24 and 24 of the second cleaning container 220 as illustrated in FIG. 7A.

Along with the movement of the magnetic bead 30, or by the moving of the cap 110 and the plunger section 130 in a direction of being drawn out from the syringe section 120 before the movement of the magnetic bead, the specimen in the adsorption solution 10 is caused to move to the syringe section 120 from the plunger section 130. The movement of the plunger section 130 causes the flow path 2 closed by the distal end portion 134 to communicate with the adsorption solution 10.

The magnetic bead 30 is lifted in the flow path 2 along with the movement of the magnet 3 and reaches the adsorption solution 10 in which the specimen is contained, as illustrated in FIG. 7B.

### (D) Process of Adsorbing Nucleic Acids to Magnetic Bead

The process of adsorbing the nucleic acids is performed by oscillating the adsorption container 100. Since the opening of the adsorption container 100 is sealed by the cap 110 such that the adsorption solution 10 does not leak out, it is possible to efficiently perform the process. Through this process, the target nucleic acids are adsorbed to the surface of the magnetic bead 30 due to the action of the chaotropic agent. In the process, in addition to the target nucleic acid, nucleic acids or protein may be attached to the surface of the magnetic bead 30.

As the method of oscillating the adsorption container 100, a known device such as a vortex shaker may be used or an operator may manually perform the mixing. In addition, a magnetic field may be externally applied using the magnetism of the magnetic bead 30 and the adsorption container 100 may be oscillated.

(E) Process of Moving of Magnetic Bead to which Nucleic Acids are Adsorbed

In the process of moving of the magnetic bead 30 to which the nucleic acids are adsorbed, the magnetic force of the magnet 3 from the outside of the adsorption container 100, the cleaning container 200, and the elution container 300 is applied to cause the magnetic bead 30 to move in the adsorption solution 10, the first to fourth oils 20, 22, 24, and 26, and the first to third cleaning solutions 12, 14, 16.

AS the magnet 3, for example, a permanent magnet, an electromagnet, or the like can be used. In addition, the magnet 3 may be caused to manually move by an operator, or the movement may be performed by using machinery equipment or the like. Since the magnetic bead 30 has properties of being attracted by the magnetic force, the magnetic bead changes relative disposition to the magnet 3 and moves inside the flow path 2 to the adsorption container 100, the cleaning container 200, and the elution container 300. There is no particular limitation to a speed when the magnetic bead 30 passes through the respective cleaning solutions and the magnetic bead 30 may move by reciprocating in the same cleaning solution in the longitudinal direction of the flow path 2. Further, in a case of causing particles other than the magnetic bead 30 to move in a tube, it is possible to perform the movement by using the gravity or a potential difference.

### (F) Process of Eluting Nucleic Acids

In the process of eluting the nucleic acids, the nucleic acids are eluted from the magnetic bead 30 in the droplet 36 of the eluate in the elution container 300. The eluate 32 in FIGS. 7A and 7B exists as the plug in the thin portions of the flow path of the elution container 300; however, the inside content expands due to the heating of the reaction container 400 during the movement of the magnetic bead 30 described above and the content moves upward in the elution container 300 as the droplet 36 as illustrated in FIGS. 8A and 8B. Also, as illustrated in FIG. 8A, when the magnetic bead 30 reaches the droplet 36 of the eluate of the elution container 300, the target nucleic acids adsorbed to the magnetic bead 30 are eluted in the droplet 36 of the eluate due to an action of the eluate.

(G) Process of Coming into Contact with Reagent 34

In the process of coming into contact with the reagent 34, the droplet 36 containing the nucleic acids is caused to come into contact with the reagent 34 positioned at the lowermost portion in the reaction container 400. Specifically, as illustrated in FIG. 8B, the cap 110 is pressed and the first oil 20 is pushed downward by the distal end portion 134 of the plunger section 130. In this manner, the magnetic bead 30, to which the magnetic force of the magnet 3 is applied, is maintained at the predetermined position, the droplet 36 of the eluate, in which the target nucleic acids are eluted, moves to the reaction container 400, and the droplet comes into contact with the reagent 34 positioned at the lowermost portion of the reaction container 400. The reagent 34, with which the droplet 36 comes into contact, is melted and mixed with the target nucleic acids in the eluate, and, for example, it is possible to perform the PCR using the thermal cycle.

### 4. PCR Device

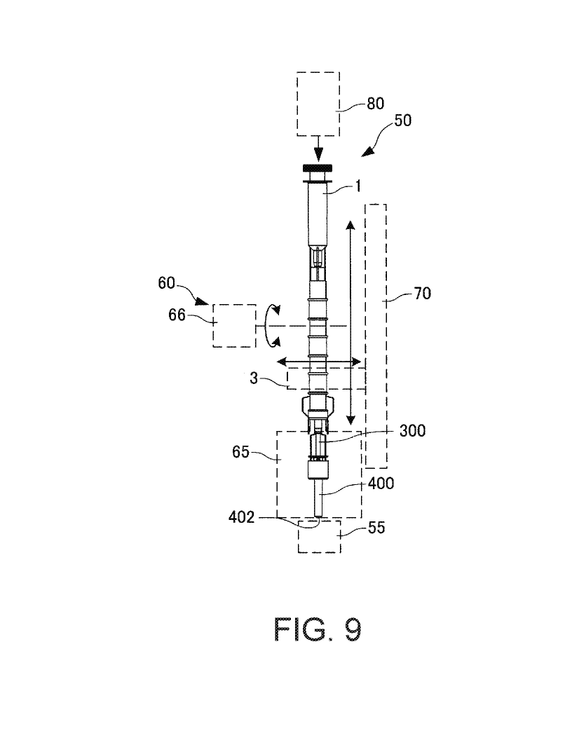

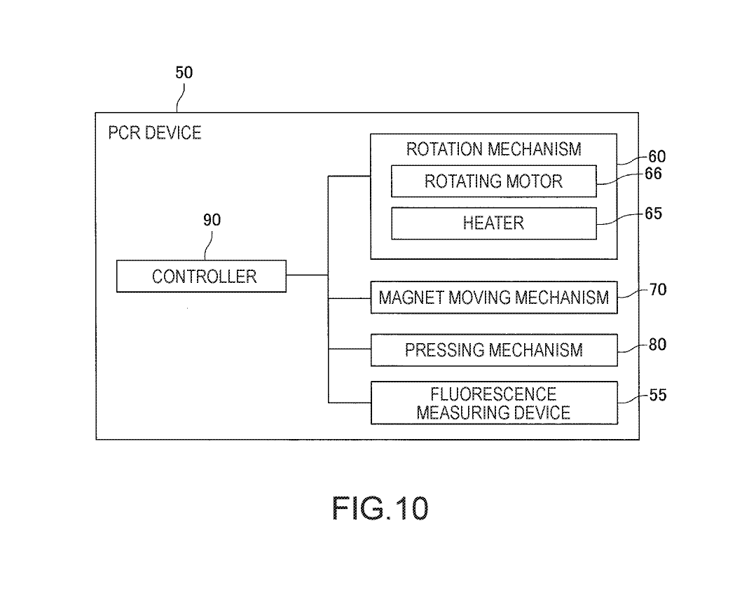

A PCR device 50 which performs nucleic acid eluting process and the PCR using the container assembly 1 is described with respect to FIG. 9 and FIG. 10. FIG. 9 is a diagram of a schematic configuration of a PCR device 50. FIG. 10 is a block diagram of the PCR device 50.

The PCR device 50 includes a rotation mechanism 60, a magnet moving mechanism 70, a pressing mechanism 80, a fluorescence measuring device 55, and a controller 90.

### 4-1. Rotation Mechanism

The rotation mechanism 60 includes a rotating motor 66 and a heater 65, and driving of the rotating motor 66 causes the container assembly 1 and the heater 65 to rotate. The rotation mechanism 60 causes the container assembly 1 and the heater 65 to rotate and to be vertically reversed, and thereby the droplet containing the target nucleic acid in the flow path of the reaction container 400 moves and the thermal cycle processing is performed.

The heater 65 includes a plurality of heaters (not illustrated), and, for example, may include a heater for elution and a high temperature and a low temperature. The eluting heater heats the eluate having the plug shape of the container assembly 1 and promotes elution of the target nucleic acids from the magnetic bead to the eluate. The high-temperature heater heats the liquid on the upstream side in the flow path of the reaction container 400 to a temperature higher than a temperature heated by the low temperature heater. The low-temperature heater heats the bottom 402 of the flow path of the reaction container. It is possible to form a temperature gradient in the liquid in the flow path of the reaction container 400 by the high-temperature heater and the low-temperature heater. A temperature control device is provided in the heater 65 and it is possible to set the temperature of the liquid in the container assembly 1, which is appropriate to a process, in response to an instruction from the controller 90.

The heater 65 has an opening through which an outer wall of the bottom 402 of the reaction container 400 is exposed. The fluorescence measuring device 55 measures the luminance of the droplet of the eluate from the opening.

### 4-2. Magnet Moving Mechanism

The magnet moving mechanism 70 is a mechanism to cause the magnet 3 to move. The magnet moving mechanism 70 attracts the magnetic bead in the container assembly 1 to the magnet 3 and causes the magnetic bead to move in the container assembly 1 by causing the magnet 3 to move. The magnet moving mechanism 70 includes a pair of magnets 3, a lifting and lowering mechanism, and an oscillating mechanism.

The oscillating mechanism is a mechanism that causes the pair of magnets 3 to oscillate in the right-left direction (or a front-rear direction in FIG. 9) in FIG. 9. The pair of magnets 3 are disposed (refer to FIG. 7A to FIG. 8B) to interpose, in the right-left direction, the container assembly 1 mounted on the PCR device 50, and it is possible to approach the magnetic bead and the magnet 3 in a direction (here, the right-left direction in FIG. 9) orthogonal to the flow path of the container assembly 1. Accordingly, when the pair of magnets 3 oscillate to follow an arrow in the right-left direction, the magnetic bead in the container assembly 1 moves in the right-left direction along with the movement. The lifting and lowering mechanism causes the magnet 3 to move in the vertical direction and it is possible to cause the magnetic bead to move in the vertical direction in FIG. 9 along with the movement of the magnet 3.

### 4-3. Pressing Mechanism

The pressing mechanism 80 is a mechanism of pressing the plunger section of the container assembly 1. The plunger section is pressed by the pressing mechanism 80, and thereby the droplet in the elution container 300 is extruded in the reaction container 400 and it is possible to perform the PCR in the reaction container 400.

In FIG. 9, the pressing mechanism 80 is disposed above the upright container assembly 1; however, a direction in which the pressing mechanism 80 presses the plunger section may not be the vertical direction in FIG. 9, but may be inclined by 45 degrees with respect to the vertical direction. In this manner, it is easy to dispose the pressing mechanism 80 at a position at which the pressing mechanism does not interfere with the magnet moving mechanism 70.

### 4-4. Fluorescence Measuring Device

The fluorescence measuring device 55 is a measuring device which measures the luminance of the droplet of the reaction container 400. The fluorescence measuring device 55 is disposed at a position facing the bottom 402 of the reaction container 400. Further, it is desirable that the fluorescence measuring device 55 can detect luminance in a plurality of wavelength bands so as to correspond to the multiplex PCRs.

### 4-5. Controller

The controller 90 is a control unit which performs control of the PCR device 50. The controller 90 includes a processor such as a CPU and a storage device such as a ROM and a RAM. Various programs and data are stored in the storage device. In addition, the storage device provides a region in which the programs are extracted. A processor executes the programs stored in the storage device, and thereby various processes are realized.

For example, the controller 90 controls the rotating motor 66 such that the container assembly 1 rotates to a predetermined rotation position. A rotation position sensor (not illustrated) is provided in the rotation mechanism 60, and the controller 90 drives and stops the rotating motor 66 in response to a detection result of the rotation position sensor.

In addition, the controller 90 controls the heater 65 such that ON/OFF control of the heater is performed, the heater generates heat, and the heater heats the liquid in the container assembly 1 to a predetermined temperature.

In addition, the controller 90 controls the magnet moving mechanism 70 such that the magnet 3 moves in the vertical direction and the magnet 3 oscillates in the right-left direction in FIG. 9 in response to the detection result of the positional sensor (not illustrated).

In addition, the controller 90 controls the fluorescence measuring device 55 and measures the luminance of the droplet in the reaction container 400. The measurement result is stored in the storage device (not illustrated) of the controller 90.

The container assembly 1 is mounted on the PCR device 50, it is possible to perform the processes of (C) to (G) in the above section 3-2, and further it is possible to perform the PCR.

### 5. Container and Liquid Storing Member

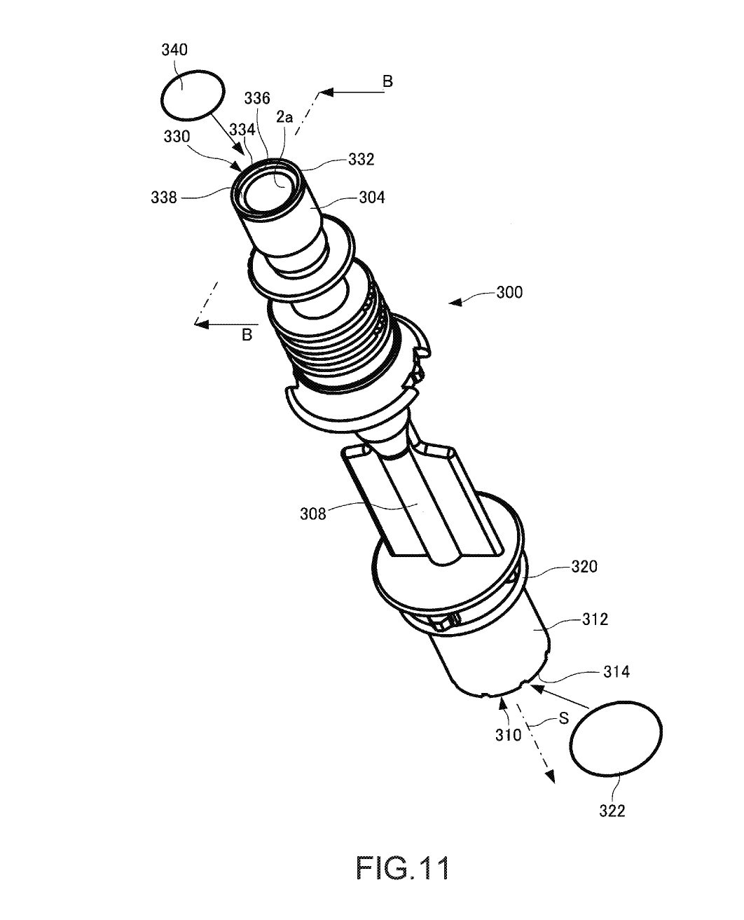

The container and the liquid storing member are described with reference to FIG. 11 to FIG. 16. As long as the liquid can be sealed and stored therein, the container is applicable to various containers; however, here, a container constituting a part of the container assembly 1 described above is described. In addition, the liquid storing member includes the container in which the liquid is sealed and stored.

### 5-1. Elution Container

The container illustrated in FIG. 11 to FIG. 13 and FIG. 16 is the elution container 300 which accommodates the eluate 32 as the liquid in a first flow path 2a and is the container described in “2-3. Elution Container” above. The elution container 300 has substantially a cylindrical shank 308 which forms a part of the first flow path 2a inside and extends in an axial direction of the first flow path 2a, and two openings 310 and 330 at both ends of the first flow path 2a.

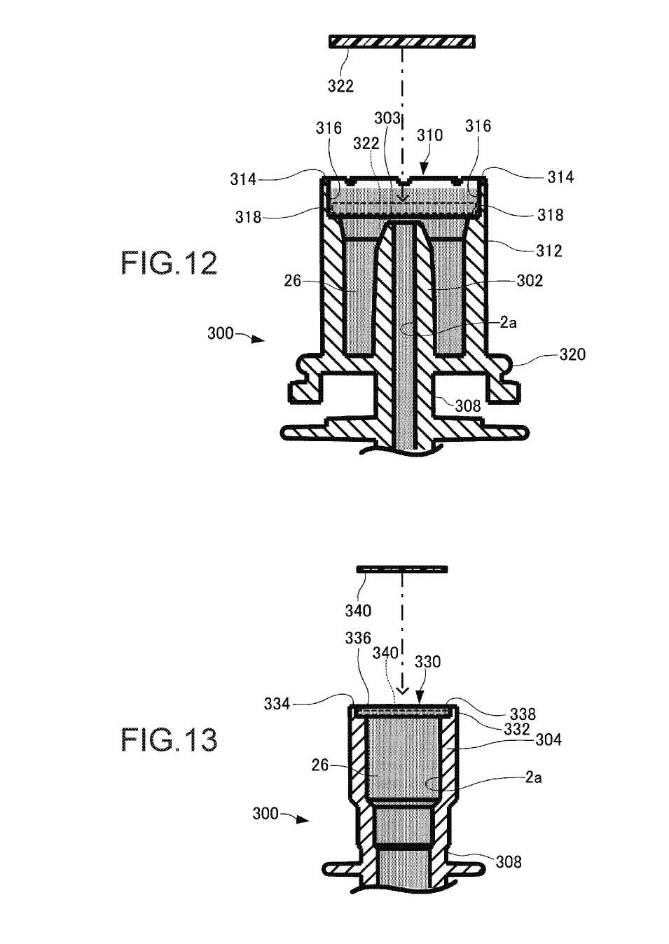

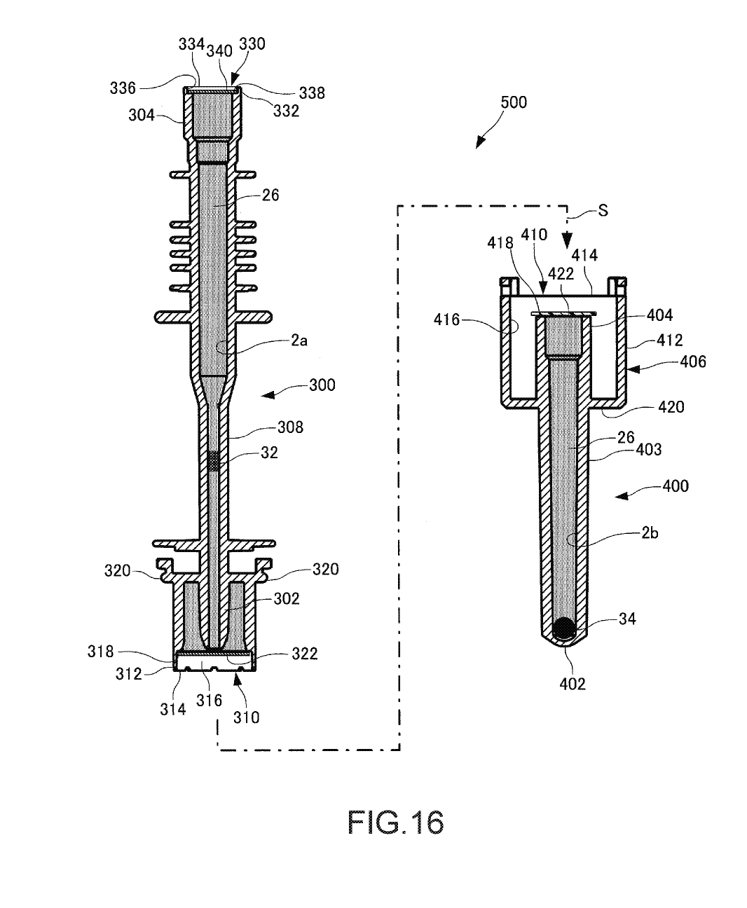

FIG. 11 is a perspective view of the elution container 300 according to the embodiment. FIG. 12 is a sectional view of a part of the elution container 300 on a first end portion 314, which is taken along line B-B. FIG. 13 is a sectional view of a part of the elution container 300 on a second end portion 334, which is taken along line B-B. FIG. 16 is a sectional view of a cartridge set 500 (the elution container 300 and the reaction container 400) according to the embodiment, which is taken along line B-B. Further, in the following description, the container and the liquid storing member are described without any particular distinction; however, the container is in a state in which no liquid is stored, and the liquid storing member has the container in which the liquid is sealed and stored.

As illustrated in FIG. 11 to FIG. 13, the elution container 300 has the two openings 310 and 330. First, with reference to FIG. 12, the first opening 310 of the elution container 300 on one end portion (first end portion 314 on the lower side in FIG. 11) is described.

As illustrated in FIG. 12, the elution container 300 has the first opening 310 and is a container in which the fourth oil 26 and the eluate 32 (FIG. 16) as the liquid are sealed and stored by sealing the first opening 310. Further, since the eluate 32 is described in “3-1-5. Eluate”, the fourth oil is described in “3-1-1. Oil”, and thus repetitive description will be omitted.

The elution container 300 has the longitudinal direction, the first opening 310 is formed at one end portion (first end portion 314) of the elution container 300, and the second opening 330 is formed at the other end portion (second end portion 334) of the elution container 300. The elution container 300 has a first annular wall section 312 which is an annular wall section having an annular wall surface formed around the first opening 310; and a first attachment surface 318 which is formed on the inner side of the first annular wall section 312 and to which a first film 322 sealing the first opening 310 is attached.

The first opening 310 has a double-cylinder structure having a cylinder on the inner side and a cylinder on the outer side, in which the cylinder on the inner side is the elution inserting section 302 and the cylinder on the outer side is the first annular wall section 312. The elution inserting section 302 and the first annular wall section 312 both have substantially a cylindrical shape. As long as the sections are tubular, any shapes may be employed.

The surface of the elution inserting section 302 on the inner side forms a part of the first flow path 2a and an opening end 303 of the elution inserting section 302 is formed at a position lower than the first attachment surface 318. Here, “high” or “low” means that the opening is on the upper side and the portion, in which the liquid is accommodated, is the downward side, unless particularly noted otherwise in the present specification, and means being high and low of a height on the upper side of the respective portions of the container in a case where the container, to which a film has yet to be attached, is filled with a liquid. The opening end 303 is positioned lower than the first attachment surface 318. Therefore, as illustrated in FIG. 12, when the filling with the fourth oil 26 is performed to the upper side of the first attachment surface 318, the opening end 303 is submerged in the liquid. Accordingly, the opening end 303 of the elution inserting section 302 does not interfere with the first film when the first film 322 is attached. In addition, the elution inserting section 302 has an outer diameter which is the same as the shank 308. An annular flange 320 protruding outward from the shank 308 is formed below the elution inserting section 302.

The first annular wall section 312 extends upward from the top surface of the flange 320. Here, “up” and “down” means the upward and downward direction in the drawings, unless particularly noted otherwise in the present specification.

The first annular wall section 312 has a height higher than the first attachment surface 318. Here, “height” means a height on the upper side of the respective portions of the container in a case where the container, to which a film has yet to be attached, is filled with a liquid, unless particularly noted otherwise in the present specification. Accordingly, in a state in which the first opening 310 faces perpendicularly upward, the top edge (first end portion 314) of the first annular wall section 312 is positioned higher than the first attachment surface 318 in comparison between the first attachment surface 318 and the first annular wall section 312. The first annular wall section 312 is positioned higher than the first attachment surface 318, and thereby it is possible to perform filling with the fourth oil 26 to a position higher than the first attachment surface 318. In this state, if an operation of attaching the first film 322 to the first attachment surface 318 is performed, it is possible to attach the first film 322 to the first attachment surface 318 in the liquid. Therefore, it is possible to prevent bubbles from being mixed to the liquid (fourth oil 26 and the eluate 32).

The first film 322 has sealing performance by which the elution container 300 can be sealed to store the liquid inside. In addition, the first film 322 has strength to the extent that the film is easily torn when binding to another container (reaction container 400) is performed.

The external appearance of the first film 322 is similar (in the present embodiment, circular shape) to the first inside surface 316 to be described below and is slightly smaller than the first inside surface. This is because it is easy to position the first film 322 to an attachment position by the first inside surface 316.

It is possible to employ a known synthetic resin film as the first film 322. In terms of thermal sealing to the first attachment surface 318, it is preferable to use a film having a polyethylene layer on its surface or, for example, it is preferable to use a film having a multi-layer structure in which a polyethylene layer is laminated on the surface of the polyester film.

The first attachment surface 318 has an annular shape and is a surface to which the first film 322 is attached. The first attachment surface 318 is an annular step section which protrudes to the inner side and is formed on the first inside surface 316 which is the wall surface of the first annular wall section 312 on the inner surface. The first attachment surface 318 is an annular flat surface and is positioned to be lower than the first end portion 314. In this manner, the first attachment surface 318 is the step section formed on the first inside surface 316, and thereby it is possible to position the first film 322 on the first inside surface 316 and to attach the first film 322 to the first attachment surface 318 when the first film 322 is attached. In other words, since the movement of the first film 322 in the horizontal direction (in FIG. 12, in the right-left and front-rear directions) is limited by the first inside surface 316, it is possible to reliably dispose the first attachment surface 318 at the predetermined attachment position.

Next, the second opening 330 of the elution container 300 on the other end portion (in FIG. 11, the second end portion 334 on the upper side) side of the elution container 300 will be described with reference to FIG. 13.

As illustrated in FIG. 13, the elution container 300 has the second opening 330 different from the first opening 310, a second annular wall section 332 having an annular wall surface formed on the periphery of the second opening 330, and a second annular attachment surface 338 to which a second film 340 sealing the second opening 330 is attached, on the inner side of the second annular wall section 332.

The second annular wall section 332 has a height higher than the second attachment surface 338. In this manner, the second annular wall section 332 and the second attachment surface 338 are also provided on the second end portion 334 side, it is possible to attach the film to the attachment surface in the liquid in both the openings 310 and 330. Therefore, it is possible to prevent bubbles from being mixed to the liquid. The second annular wall section 332 is a part of the upper end of the elution receiving section 304.

The elution receiving section 304 is a portion in which the third insertion section 232 of the third cleaning container 230 is received as described in “2-3. Elution Container”.

The second attachment surface 338 is an annular step section which protrudes to the inner side and is formed on a second inside surface 336 which is the annular inside surface of the elution receiving section 304. The second attachment surface 338 has an annular shape and is a surface to which the second film 340 is attached. The second attachment surface 338 is an annular flat surface and is positioned to be lower than the second end portion 334. In this manner, the second attachment surface 338 is the step section formed on the second inside surface 336, and thereby it is possible to position the second film 340 on the second inside surface 336 and to attach the second film to the second attachment surface 338 when the second film 340 is attached.

As the second film 340, it is possible to employ a film having the same function as the first film 322.

### 5-2. Liquid Storing Member

As illustrated on the left side in FIG. 16, the elution container 300 in which the liquid (fourth oil 26 and eluate 32) is sealed and stored corresponds to the liquid storing member.

The first film 322 is attached to the first attachment surface 318 of the elution container 300, the second film 340 is attached to the second attachment surface 338, and the liquid (fourth oil 26 and eluate 32) is sealed and stored in the liquid storing member in the elution container 300 between the first film 322 and the second film 340. As described in “5-1. Elution Container” above, according to the liquid storing member, it is possible to seal and store the liquid in a state in which bubbles are less likely to be mixed into the liquid. Further, elution container 300 has two openings, and thus has such a configuration described above; however, the reaction container 400 and the adsorption container 100 to be described below may have a structure in which the film is attached only on one opening on a side on which another container is bound.

In the liquid storing member, the elution inserting section 302 and the opening end 303 thereof are in the liquid of the fourth oil 26.

### 5-3. Method of Manufacturing of Liquid Storing Member

A method of manufacturing the liquid storing member is described with reference to FIG. 11 to FIG. 13.

First, as illustrated in FIG. 11, the elution container 300, two openings 310 and 330 of which are not sealed, is prepared.

Next, as illustrated in FIG. 13, the second opening 330 faces perpendicularly upward and the second film 340 is mounted on the second attachment surface 338. Since the movement of the second film 340 in the horizontal direction (in FIG. 13, in the right-left and front-rear directions) is limited by the second inside surface 336, it is possible to easily dispose the second film at the predetermined position. Further, at this time, the first opening 310 is not sealed, and thus the fourth oil 26 does not exist unlike in FIG. 13. Filling with the fourth oil 26 as in FIG. 13 is performed in a case where the first opening 310 is, at first, sealed. Further, the vicinity of the outer edge of the second film 340 is pressed on the second attachment surface 338 and is heated, and thereby the second film 340 is attached to the second attachment surface 338.

Next, as illustrated in FIG. 12, the first opening 310 faces perpendicularly upward, the liquid (the fourth oil 26, the eluate 32, and the fourth oil 26, entering in this order) is injected into the elution container 300, and filling with the liquid (fourth oil 26) is performed to a level higher than the first attachment surface 318. At this time, the first attachment surface 318 is positioned lower than the surface of the fourth oil 26 and is submerged in the liquid.

In this state, the first film 322 is mounted on the first attachment surface 318 in the liquid (position of a dotted line in the drawings). Since the movement of the first film 322 in the horizontal direction (in FIG. 13, in the right-left and front-rear directions) is limited by the first inside surface 316, it is possible to easily dispose the first film at the predetermined position. Also, the vicinity of the outer edge of the first film 322 is pressed on the first attachment surface 318 and is heated to adhere to each other, and thereby the first film 322 is attached to the first attachment surface 318 and the liquid (fourth oil 26 and eluate 32) is stored in the sealed state. In this manner, the first film 322 is attached in the liquid, and thereby it is possible to prevent bubbles from being mixed in the liquid with which the filling is performed. A method of attachment of the first film 322 is preferably the adhesion; however, the attachment is not limited to the adhesion, but it is possible to employ another known attachment method.

In this manner, the elution container 300, in which the fourth oil 26 and the eluate 32 are sealed and stored in the first flow path 2a, is a liquid storing member in a state illustrated on the left side in FIG. 16. Further, an example, in which sealing is performed on the second opening 330 side in advance, is described; however, the sealing may be performed on the first opening 310 side in advance. In this case, similar to the first opening 310 described here, after the liquid is injected higher than the second attachment surface 338, the second film 340 is attached.

### 5-4. Reaction Container

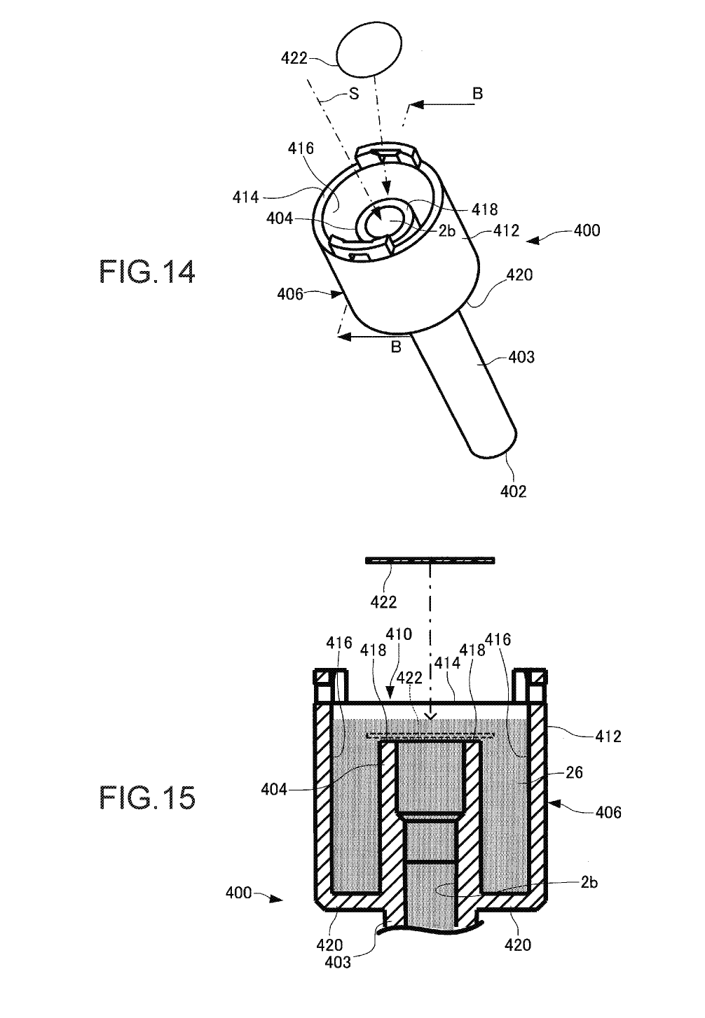

The container illustrated in FIG. 14 to FIG. 16 is the reaction container 400 in which the fourth oil 26 as the liquid is sealed and stored by sealing the third opening 410 and corresponds to the container described in “2-4. Reaction Container” above. FIG. 14 is a perspective view of the reaction container 400 according to the embodiment. FIG. 15 is a sectional view of a part of the reaction container 400 on the third end portion 414 side, which is taken along line B-B.

The reaction container 400 has a cylindrical shank 403 which forms a second flow path 2b inside, a third opening 410 formed on one end portion, the bottom 402 which closes the second flow path 2b formed at the other end portion, the cylindrical reservoir section 406 formed on the periphery of the shank 403 on the third opening 410 side, and a reaction receiving section 404 in which the elution inserting section 302 is received in the second flow path 2b. The second flow path 2b contains the fourth oil 26 and the reagent 34 which reacts with the nucleic acids eluted in the eluate 32.

The reaction container 400 has a third annular wall section 412 having an annular wall surface on the third end portion 414 side, as one end portion, and a third annular attachment surface 418 which is formed on the inner side of the third annular wall section 412 and to which a third film 422 sealing the third opening 410 is attached.

Similar to the first opening 310, the third opening 410 has the double-cylinder structure in which the cylinder on the inner side is the reaction receiving section 404 and the cylinder on the outer side is the third annular wall section 412. When the elution container 300 is bound to the reaction container 400, a space between the outside surfaces of the third annular wall section 412 and the reaction receiving section 404 can receive a liquid which leaks out from the flow paths 2a and 2b such that the liquid does not leak to the outside with the first film 322 and the third film 422 being torn.

The inner surface of the reaction receiving section 404 forms a part of the second flow path 2b and has an outer diameter which is substantially the same as the shank 403. An annular connection section 420 protruding outward from the shank 308 is formed below the reaction receiving section 404. The third annular wall section 412 extends upward from the outer circumferential edge of the connection section 420. The reaction receiving section 404 and the third annular wall section 412 have both substantially a cylindrical shape. However, as long as the sections are tubular, any shapes may be employed.

The third attachment surface 418 has an annular shape and is a surface to which the third film 422 is attached. The third attachment surface 418 is the top surface of the reaction receiving section 404. The third attachment surface 418 is an annular flat surface.

The third annular wall section 412 has a height higher than the third attachment surface 418. Accordingly, in a state in which the third opening 410 faces perpendicularly upward, the top edge (third end portion 414) of the third annular wall section 412 is positioned higher than the third attachment surface 418, in comparison between the third attachment surface 418 and the third annular wall section 412. The third annular wall section 412 is positioned higher than the third attachment surface 418, and thereby it is possible to perform filling with the fourth oil 26 to a position higher than the third attachment surface 418. In this state, if an operation of attaching the third film 422 to the third attachment surface 418 is performed, it is possible to attach the third film 422 to the third attachment surface 418 in the liquid. Therefore, it is possible to prevent bubbles from being mixed to the fourth oil 26.

As the third film 422, it is possible to employ a film having the same function as the first film 322. The third film 422 has the external appearance of a circular shape and has an outer diameter greater than that of the third attachment surface 418. In a state in which the third film 422 is attached to the third attachment surface 418, the outer circumferential edge of the third film 422 has a gap with the third inside surface 416.

### 5-5. Another Liquid Storing Member

As illustrated in FIG. 16, the reaction container 400 in which another liquid (fourth oil 26) is sealed and stored corresponds to another liquid storing member. In the other liquid storing member, the third film 422 is attached to the third attachment surface 418 of the reaction container 400, and the liquid (fourth oil 26) is sealed and stored in the reaction container 400. As described in “5-4. reaction container” above, according to the other liquid storing member, it is possible to perform sealing to store the liquid in a state in which bubbles are less likely to be mixed in the liquid.

### 5-6. Method of Manufacturing of Liquid Storing Member

A method of manufacturing the other liquid storing member is described with reference to FIG. 14 and FIG. 15.

First, as illustrated in FIG. 14, the reaction container 400, the third opening 410 of which is not sealed, is prepared.

Next, as illustrated in FIG. 15, the third opening 410 faces perpendicularly upward, the liquid (the reagent 34 and the fourth oil 26) is injected into the reaction container 400, and filling with the liquid (fourth oil 26) is performed to a level higher than the third attachment surface 418. At this time, the third attachment surface 418 is positioned lower than the surface of the fourth oil 26 and is submerged in the liquid.

The third film 422 is mounted on the third attachment surface 418 in the liquid (position of a dotted line in the drawings). Also, the vicinity of the outer edge of the third film 422 is pressed on the third attachment surface 418 and is heated, and thereby the third film 422 is attached to the third attachment surface 418 such that the liquid (fourth oil 26 and reagent 34) is stored in the sealed state. In this manner, the third film 422 is attached in the liquid, and thereby it is possible to prevent bubbles from being mixed in the stored liquid in the sealed state.

In this manner, the reaction container 400, in which the fourth oil 26 and the reagent 34 are sealed and stored in the second flow path 2b, is a liquid storing member in a state illustrated on the right side in FIG. 16.

### 6. Cartridge Set

The cartridge set 500 is described with reference to FIG. 16. FIG. 16 is a sectional view of the cartridge set 500 according to an embodiment, which is taken along line B-B.

As illustrated in FIG. 16, the cartridge set 500 includes one liquid storing member as the elution container 300 described in “5-2. Liquid Storing Member” above and the other liquid storing member as the reaction container 400 described in “5-5. Another Liquid Storing Member” above, which is bound to the liquid storing member.

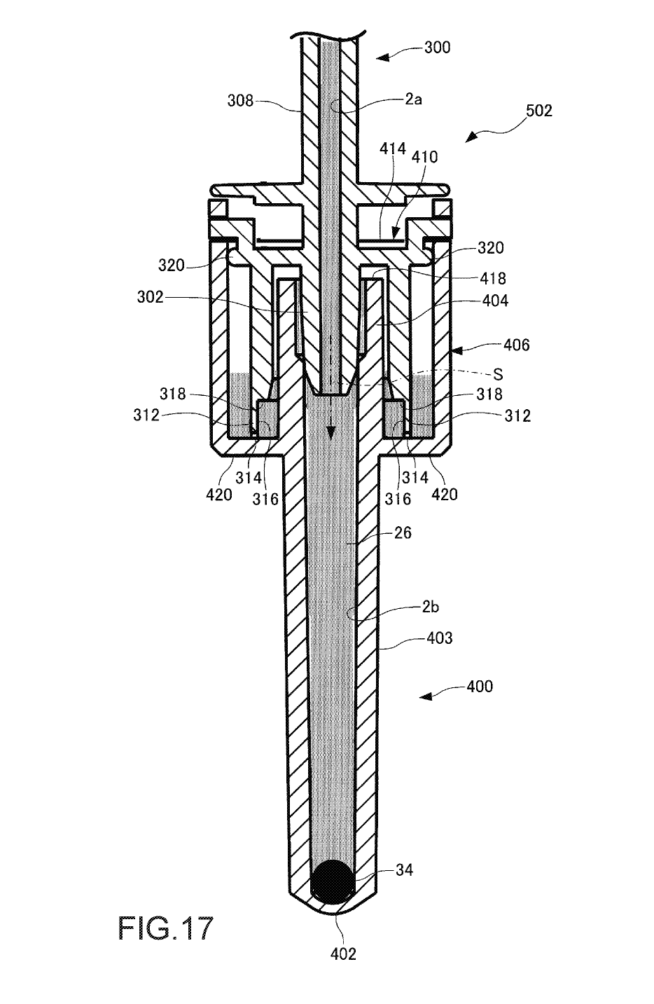

The liquid storing member as the elution container 300 can be bound to the other liquid storing member as the reaction container 400 such that the elution inserting section 302 is inserted into the reaction receiving section 404 in an insertion direction S. Also, at the time of a binding operation, the first film 322 and the third film 422 are torn by the opening end 303 of the elution inserting section 302 and the third attachment surface 418 of the reaction receiving section 404. Since the opening end 303 of the elution inserting section 302 is inserted in the reaction receiving section 404 which is fully filled with the fourth oil 26, in the liquid as is, it is difficult for bubbles to be mixed into between (a flow path which is formed to communicate with the two flow paths) the first flow path 2a and the second flow path 2b even during the binding operation. The binding state is illustrated in FIG. 17. FIG. 17 is a sectional view of a part of a nuclei acid amplifying reaction cartridge 502, which is taken along line B-B. Further, in FIG. 17, the film is not shown.

In addition, as described above, according to the cartridge set 500, it is possible to bind two liquid storing member, in which the liquids are sealed and stored in the state in which bubbles are less likely to be mixed therein, and it is possible to assemble the nuclei acid amplifying reaction cartridge 502.

The cartridge set 500 (FIG. 16) described above is a set of the elution container 300 and the reaction container 400, but can be assembled with an appropriate combination of the other containers (100, 200, 300, and 400) described above. The sealing structure of both the openings of the elution container 300 can be similarly employed to the first cleaning container 210, the second cleaning container 220, and the third cleaning container 230. In the case of the first and second cleaning containers 210 and 220, a plate-shaped member protrudes from the first and second insertion sections 212 and 222 (FIG. 6); however, similarly, a film is attached to the plate-shaped member as in the liquid. In addition, it is possible to employ the sealing structure in the first opening 310 of the elution container 300 to the end portion which is bound to the first cleaning container 210 in the adsorption container 100 and it is possible to insert the plunger section 130, to which the film is attached, into the other opening. Also, these containers (100, 200, 300, and 400) are bound, and thereby it is possible to obtain the container assembly 1 described above. Further, it is possible to appropriately alter the binding process order of the respective containers and it is possible to appropriately alter the combination between the containers.

The invention is not limited to the embodiments described above and further can be variously modified. For example, the invention includes substantially the same configuration (for example, configuration having the same function, method, and result, or configuration having the same object and effects) as the configuration described in the embodiments. In addition, the invention includes a configuration in which a portion, which is not a fundamental portion of the configuration described in the embodiments, is replaced. In addition, the invention includes a configuration in which the same effect is achieved, or a configuration in which the same object is achieved, as that of the configuration described in the embodiments. In addition, the invention includes a configuration in which a known technology is added to the configuration described in the embodiments.

The entire disclosure of Japanese Patent Application No. 2015-031625, filed Feb. 20, 2015 is expressly incorporated by reference herein.

What is claimed is:

1. A container that has an opening and in which a liquid is sealed and stored by sealing the opening, the container, comprising:
an annular wall section having an annular wall surface formed around the opening; and
an attachment surface which is formed on the inner side of the annular wall section and to which a film sealing the opening is attached,
wherein the annular wall section has a height higher than the attachment surface.

2. The container according to claim 1,
wherein the attachment surface is an annular step section formed on a wall surface on the inner side of the annular wall section.

3. The container according to claim 1, in which the opening, the annular wall section, the film, and the attachment surface are a first opening, a first annular wall section, a first film, and a first attachment surface, respectively, the container further comprising:
a second opening different from the first opening;
a second annular wall section having an annular wall surface formed around the second opening; and
a second annular attachment surface which is formed on the inner side of the second annular wall section and to which a second film sealing the second opening is attached,
wherein the second annular wall section has a height higher than the second attachment surface.

4. The container according to claim 3,
wherein the container has a longitudinal direction,
wherein the first opening is formed in one end portion of the container, and
wherein the second opening is formed in the other end portion of the container.

5. A liquid storing member comprising the container according to claim 1 to which the film is attached on the attachment surface thereof and in which a liquid is sealed and stored.

6. A liquid storing member comprising the container according to claim 2 to which the film is attached on the attachment surface thereof and in which a liquid is sealed and stored.

7. A liquid storing member comprising the container according to claim 3 to which the first film is attached on the first attachment surface thereof and the second film is attached on the second attachment surface thereof and in which a liquid is sealed and stored between the first film and the second film.

8. A liquid storing member comprising the container according to claim 4 to which the first film is attached on the first attachment surface thereof and the second film is attached on the second attachment surface thereof and in which a liquid is sealed and stored between the first film and the second film.

9. A cartridge set comprising:
the liquid storing member according to claim 5; and
another liquid storing member which is bound to the liquid storing member,
wherein a liquid is sealed and stored in a first flow path of the liquid storing member and the liquid storing member further includes an insertion section which is inserted into the another liquid storing member,
wherein an inside surface of the insertion section forms a part of the first flow path,
wherein an opening end of the insertion section is formed at a position lower than the attachment surface,
wherein the another liquid storing member has a second flow path that stores another liquid inside, a third annular wall section having an annular wall surface on one end portion side, a third annular attachment surface that is formed on the inner side of the third annular wall section and to which a third film is attached,
wherein the third annular wall section has a height higher than the third attachment surface, and
wherein the third film is attached to the third attachment surface and the another liquid is sealed and stored in the second flow path.

10. A cartridge set comprising:
the liquid storing member according to claim 6; and
another liquid storing member which is bound to the liquid storing member,
wherein a liquid is sealed and stored in a first flow path of the liquid storing member and the liquid storing member further includes an insertion section which is inserted into the another liquid storing member,
wherein an inside surface of the insertion section forms a part of the first flow path,
wherein an opening end of the insertion section is formed at a position lower than the attachment surface,
wherein the another liquid storing member has a second flow path that stores another liquid inside, a third annular wall section having an annular wall surface on one end portion side, a third annular attachment surface that is formed on the inner side of the third annular wall section and to which a third film is attached,
wherein the third annular wall section has a height higher than the third attachment surface, and
wherein the third film is attached to the third attachment surface and the another liquid is sealed and stored in the second flow path.

11. A cartridge set comprising:
the liquid storing member according to claim 7; and
another liquid storing member which is bound to the liquid storing member,
wherein a liquid is sealed and stored in a first flow path of the liquid storing member and the liquid storing member further includes an insertion section which is inserted into the another liquid storing member,
wherein an inside surface of the insertion section forms a part of the first flow path,
wherein an opening end of the insertion section is formed at a position lower than the attachment surface,
wherein the another liquid storing member has a second flow path that stores another liquid inside, a third annular wall section having an annular wall surface on one end portion side, a third annular attachment surface that is formed on the inner side of the third annular wall section and to which a third film is attached,
wherein the third annular wall section has a height higher than the third attachment surface, and
wherein the third film is attached to the third attachment surface and the another liquid is sealed and stored in the second flow path.

12. A cartridge set comprising:
the liquid storing member according to claim 8; and
another liquid storing member which is bound to the liquid storing member,
wherein a liquid is sealed and stored in a first flow path of the liquid storing member and the liquid storing member further includes an insertion section which is inserted into the another liquid storing member,
wherein an inside surface of the insertion section forms a part of the first flow path,
wherein an opening end of the insertion section is formed at a position lower than the attachment surface,
wherein the another liquid storing member has a second flow path that stores another liquid inside, a third annular wall section having an annular wall surface on one end portion side, a third annular attachment surface that is formed on the inner side of the third annular wall section and to which a third film is attached,
wherein the third annular wall section has a height higher than the third attachment surface, and
wherein the third film is attached to the third attachment surface and the another liquid is sealed and stored in the second flow path.

13. A method of manufacturing a liquid storing member comprising:
injecting a liquid into the container according to claim 1;
filling the container with the liquid at a level higher than the attachment surface; and
attaching the film to the attachment surface in the liquid to seal and store the liquid.

14. A method of manufacturing a liquid storing member comprising:
injecting a liquid into the container according to claim 2;
filling the container with the liquid at a level higher than the attachment surface; and
attaching the film to the attachment surface in the liquid to seal and store the liquid.
